(19) United States
(12) Patent Application Publication
(10) Pub. No.: US 2024/0112750 A1
(43) Pub. Date: Apr. 4, 2024

(54) DATA-DRIVEN PROCESS DEVELOPMENT AND MANUFACTURING OF BIOPHARMACEUTICALS

(71) Applicant: BioCurie Inc., Wilmington, DE (US)

(72) Inventors: Richard D. Braatz, Arlington, MA (US); Irene Rombel, Hockessin, DE (US)

(21) Appl. No.: 17/959,537

(22) Filed: Oct. 4, 2022

(51) Int. Cl. G16B 5/00; G06N 5/02; G16B 20/00; G16B 40/20

(52) U.S. Cl. CPC G16B 5/00; G06N 5/022; G16B 20/00; G16B 40/20

(57) ABSTRACT

Disclosed is a method implemented for outputting model(s) for developing or operating a process for a CGT. The method includes receiving, storing, and accessing data items; determining attributes of the data items; selecting one or more machine learning models based on the attributes; accessing one or more mechanistic models; integrating the one or more machine learning models with the one or more mechanistic models to obtain one or more integrated models; selecting one or more predictive models from the one or more machine learning models, the one or more mechanistic models, and the one or more integrated models; applying the one or more predictive models to the data items; adjusting one or more values of one or more parameters of the one or more predictive models to reduce uncertainty in model prediction; and outputting the one or more predictive models with the one or more adjusted values.

### BACKGROUND

A biopharmaceutical—also known as a biological medical product, biotherapeutic, or biologic—is any pharmaceutical drug product manufactured in, extracted from, or semi synthesized from biological sources. The production of these biopharmaceuticals involves complex process development and manufacturing, largely due to the uncertainties at almost every stage of the development and the manufacturing. Here, process development means development of a robust, scalable, and reproducible process with the goal of producing safe and efficacious biopharmaceuticals in a cost-effective manner, and manufacturing means production of biopharmaceuticals for clinical trial or commercial supply.

One area of focus in modern biopharmaceutics is cell or gene therapy (CGT), which further includes sub-areas such as cell therapy (CT), gene therapy (GT), nucleic acid (NA) therapies and vaccines, and regenerative medicine. In particular, CT generally refers to ex vivo production of cells and delivery to a human subject, or in vivo production of cells in a human subject, to achieve a therapeutic or preventive effect. CT may or may not involve gene modification. GT generally refers to in vivo delivery of a gene or genetic element to a human subject to achieve a therapeutic or preventive effect.

### SUMMARY

In accordance with one aspect of the present disclosure, a method implemented by a data processing system is provided for outputting one or more models for developing or operating a process for a CGT. The method includes receiving a plurality of data items, storing the plurality of data items on a hardware storage device, and accessing the plurality of data items using the data processing system. The method includes determining, by the data processing system, one or more attributes of the plurality of data items and selecting one or more machine learning models based on the one or more attributes. The method includes accessing one or more mechanistic models. The method includes integrating, by the data processing system, the one or more machine learning models with the one or more mechanistic models to obtain one or more integrated models and selecting one or more predictive models from the one or more machine learning models, the one or more mechanistic models, and the one or more integrated models. The method includes applying the one or more predictive models to the plurality of data items. The method includes adjusting one or more values of one or more parameters of the one or more predictive models to reduce uncertainty in model prediction. The method includes outputting, by the data processing system, the one or more predictive models with the one or more adjusted values of the one or more parameters.

In accordance with one aspect of the present disclosure, a non-transitory computer-readable medium is provided. The non-transitory computer-readable medium contains program instructions that, when executed, cause a data processing system to perform operations for developing or operating a process for a CGT. The operations include receiving a plurality of data items, storing the plurality of data items on a hardware storage device, and accessing the plurality of data items. The operations include determining one or more attributes of the plurality of data items and selecting one or more machine learning models based on the one or more attributes. The operations include accessing one or more mechanistic models. The operations include integrating the one or more machine learning models with the one or more mechanistic models to obtain one or more integrated models and selecting one or more predictive models from the one or more machine learning models, the one or more mechanistic models, and the one or more integrated models. The operations include applying the one or more predictive models to the plurality of data items. The operations include adjusting one or more values of one or more parameters of the one or more predictive models to reduce uncertainty in model prediction. The operations include outputting the one or more predictive models with the one or more adjusted values of the one or more parameters.

In some implementations of the method or the non-transitory computer-readable medium, the one or more attributes include at least one of nonlinearity, non-normality, collinearity, or dynamics.

In some implementations of the method or the non-transitory computer-readable medium, the one or more mechanistic models are accessed based on at least one of a physical property, a chemical property, or a biological property of the process.

In some implementations of the method or the non-transitory computer-readable medium, integrating the one or more machine learning models with the one or more mechanistic models includes: arranging the one or more machine learning models and the one or more mechanistic models in a sequence comprising a first one or more models and a second one or more models; transmitting an output of the first one or more models to the second one or more models; transmitting data to the second one or more models; and obtaining an output of the second one or more models.

In some implementations of the method or the non-transitory computer-readable medium, integrating the one or more machine learning models with the one or more mechanistic models includes: determining a first one or more models and a second one or more models from the one or more machine learning models and the one or more mechanistic models; transmitting input data to the first one or more models; constraining a prediction of the first one or more models using the second one or more models; and obtaining an output of the first one or more models.

Implementations of the method or the non-transitory computer-readable medium can be applied to a variety of CGT techniques.

### BRIEF DESCRIPTION OF THE DRAWINGS

FIG. 1 provides an example flow for uncertainty reduction in process development and manufacturing for CGT, according to some implementations.

FIG. 2 provides an example block diagram illustrating the selection and integration of models, according to some implementations.

FIG. 3A provides an example chart illustrating the selection of machine learning models, according to some implementations.

FIG. 3B illustrates an example biomanufacturing process in which modelling is applied, according to some implementations.

FIGS. 4A-4J each illustrate an example mechanism for integrating one or more machine learning models with one or more mechanistic models, according to some implementations.

FIG. 5 illustrates example CGT modalities, according to some implementations.

FIG. 6 illustrates example techniques involved in CGT, according to some implementations.

FIG. 7 illustrates a flowchart of an example method, according to some implementations.

FIG. 8 illustrates a block diagram of an example computer system, according to some implementations.

Figures are not drawn to scale. Like reference numbers refer to like components.

### DETAILED DESCRIPTION

Process development and manufacturing of biopharmaceuticals have significant impact on the safety, efficacy, scalability, and cost of medicinal products, particularly for CGTs owing to their high complexity. The nature of uncertainties in the process development and the manufacturing calls for a mechanism to leverage data, analytics, and modeling for prediction and optimization.

Machine learning is often regarded as a potential solution for making predictions after being trained with sample data of similar kinds. Commonly adopted machine learning techniques include supervised and unsupervised learning, neural networks, natural language processing, symbolic reasoning, algebraic learning, support vector machine, ensemble methods, kernel methods, k-nearest neighbors, automatic learning, reinforcement learning, and Bayesian optimization. While machine learning techniques have been widely adopted to describe systems in other industries, these techniques have thus far been rarely adopted in the biopharmaceutical industry. Among the very few applications of machine learning in the biopharmaceutical industry, most are limited to describing small molecules and recombinant protein-based therapeutics such as antibodies. For process development and manufacturing of complex biopharmaceuticals such as CGT, sole machine learning techniques face the challenge of not having training samples with adequate quantity and quality for making predictions.

In light of the above problems, this disclosure provides one or more mechanisms that integrate a machine learning model with a mechanistic model. In this disclosure, a model generally refers to a description of a system using mathematical concepts and language. A model describes a system by a set of variables and a set of equations that establish relationships between the variables. A model can be used to explain a system, study the effects of components of the system, and make predictions. A model can be implemented as software code on a computer.

Different from a machine learning model that relies on input sample data to make predictions, a mechanistic model is made based on the application of physical, chemical, and/or biological properties that describe the behavior of constituting parts of the modeled system. For example, a mechanistic model may receive input parameters on the raw materials to a bioreactor and apply mathematical expressions describing the underlying biological processes to predict the production rate and quality of the produced biopharmaceutical. The mechanistic model may describe phenomena that are intracellular and/or extracellular and/or involve multiple cell populations, and may be informed from process and/or omic data, e.g., genomic, transcriptomic, proteomic, epigenomic, metabolomic, fluxomic, glycomic. The mechanistic model may describe phenomena occurring in liquid or solid solutions, in single or multiple phases, or on surfaces, e.g., such as for nucleic acids (e.g., oligonucleotides) produced by solid-phase synthesis. By integrating the machine learning model with the mechanistic model, implementations of the disclosure allow for data-driven prediction for reducing uncertainties in complex process development and manufacturing of biopharmaceuticals.

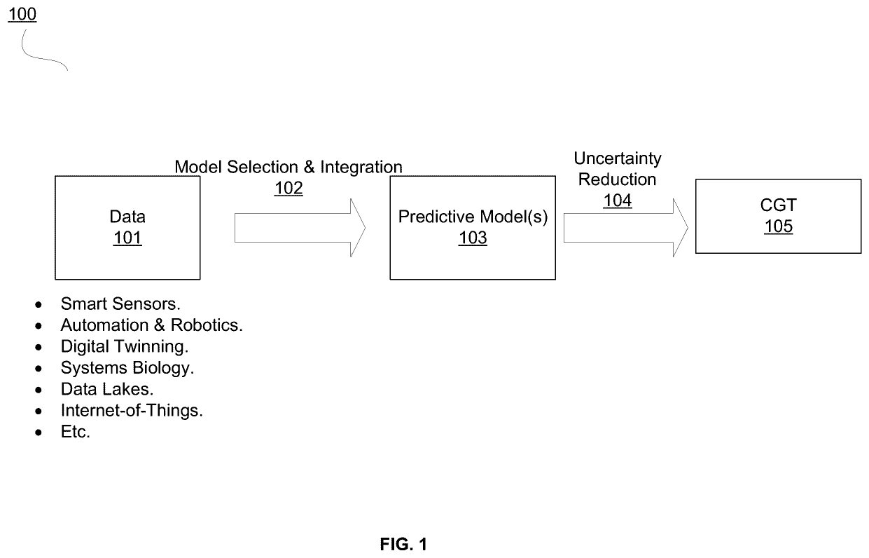

FIG. 1 illustrates a flow 100 of uncertainty reduction during process development and manufacturing for CGT, according to some implementations. As shown in FIG. 1, flow 100 receives input data 101, which are used to form one or more predictive models 103. Predictive models 103 are then applied to the specific process development or manufacturing to reduce uncertainty in predictions for CGT 105.

In FIG. 1, data 101 can be collected from a variety of sources, such as physical sensors (pressure, temperature), chemical sensors (e.g., pH, pIon, metabolites), smart sensors, spectral sensors (e.g., Raman, fluorescence, near-infrared), imaging instruments (e.g., microscopy, holography, hyperspectral), assays, automation and robotics, digital twinning, systems biology, data lakes, internet-of-things, etc. In some implementations, data 101 include sample data for training a machine learning model. In some implementations, data 101 are updated periodically or spontaneously as flow 100 reduces uncertainties in the predictions for CGT.

At operation 102, flow 100 involves model selection and/or integration based on data 101. Two types of models are involved at operation 102: one or more machine learning models; and one or more mechanistic models. After the machine learning models and the mechanistic models are selected and accessed, the two types of models are integrated to become one or more predictive models 103.

In some implementations, predictive models 103 make predictions to reduce uncertainty at operation 104. The uncertainty can be mathematically represented and adjusted in a number of ways, such as parameter-adaptive extended Kalman filtering, parameter-adaptive Luenberger observers, and Bayesian adaptive ensemble Kalman filtering. The parameters involved in operation 104 can be extracted from input data 101. Predictive models 103 can be used in a wide range of applications, including design of the control strategy, maximizing production, scale-up of production, technology transfer of production (e.g., to different sites), process monitoring, root cause analysis (e.g., troubleshooting), and economic modeling.

Predictive models 103 can be used in various applications of CGT 105, with scales ranging from 1 mL per production run to 25,000 L per production run. In addition, the production can be automated or semi-automated and can be an open, semi-closed, or closed system. In addition, the production can be automated for one or more unit operations, or for an end-to-end production system or facility.

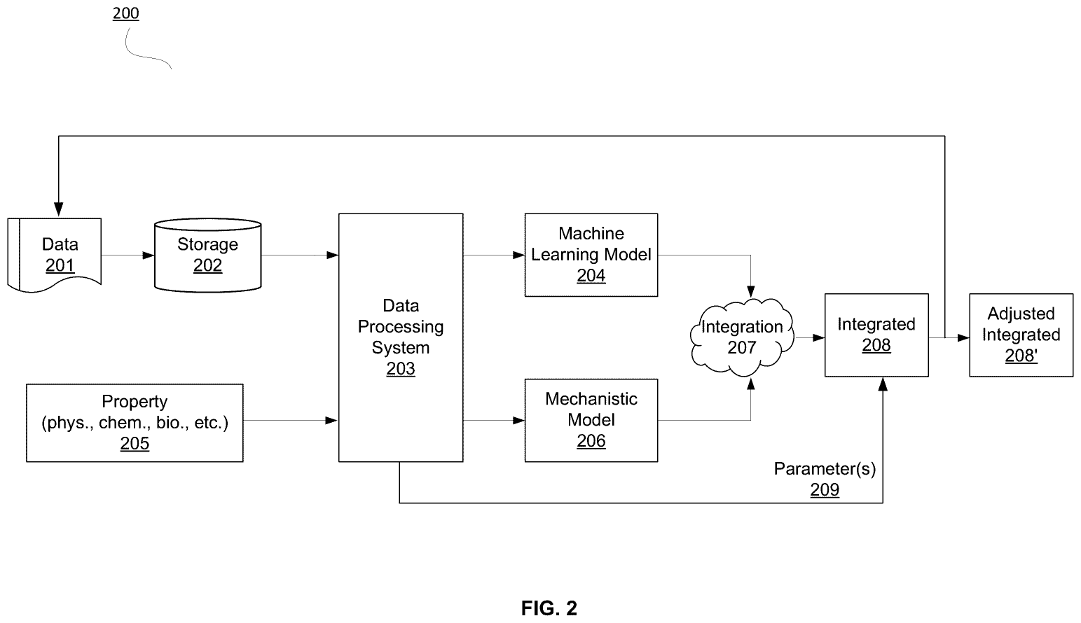

FIG. 2 provides an example block diagram illustrating model selection and integration operations 200, according to some implementations. Operations 200 may correspond fully or in part with operation 102 of FIG. 1. In some implementations, operations 200 are executed by computer hardware including, e.g., data processing system 203 and storage 202. In this example, storage 202 includes a non-transitory hardware storage device that in combination with memory of data processing system 203 causes data processing system 203 to perform the functionality described herein.

According to FIG. 2, operations 200 include receiving input data 201 and saving the same in storage 202. Similar to data 101 of FIG. 1, data 201 of FIG. 2 can include sample data for training a machine learning model. By accessing data 201 from storage 202, data processing system 203 can select a machine learning model 204.

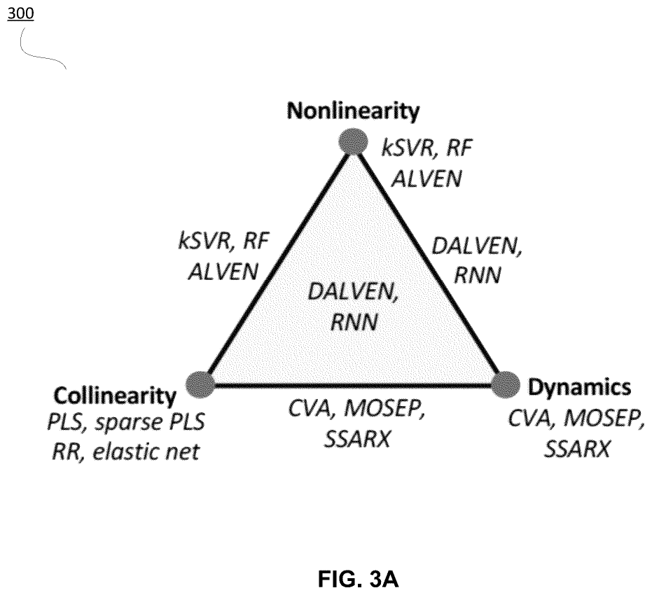

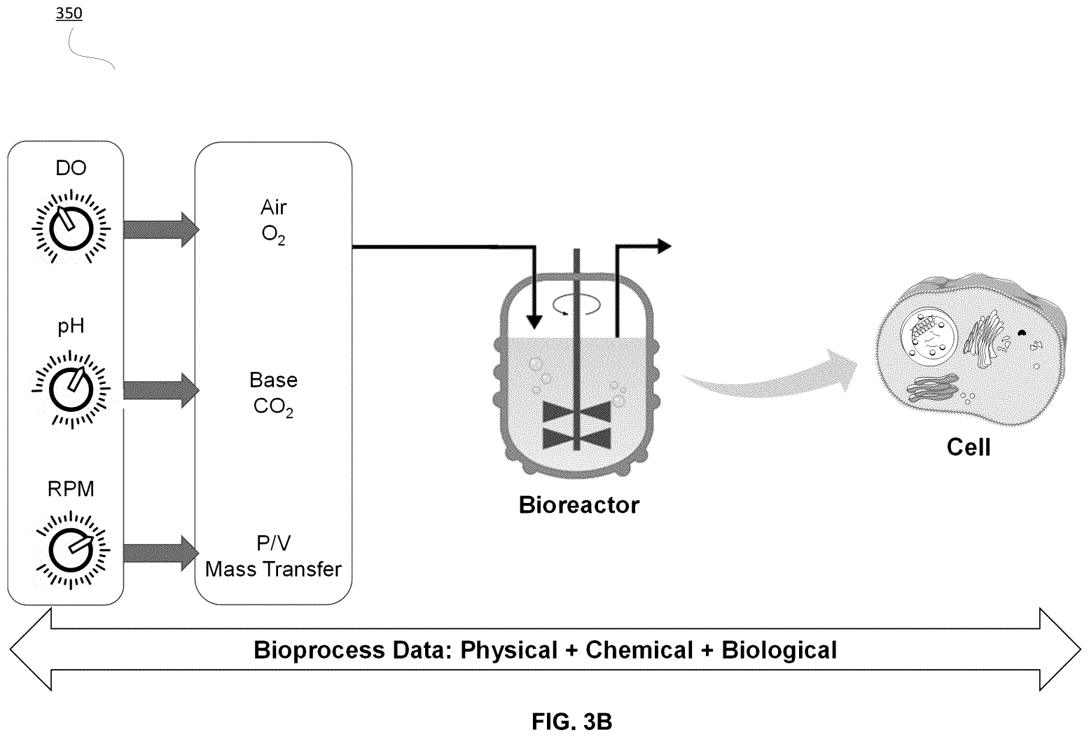

In some implementations, the selection of machine learning model 204 is based on one or more attributes of data 201. Specifically, after accessing data 201 from storage 202, data processing system 203 determines one or more attributes on data 201 and makes the selection based on the one or more attributes. The selection is described later in detail with reference to FIG. 3A.

In addition to selecting machine learning model 204, data processing system 203 accesses a mechanistic model 206. In some implementations, the access is based on a physical property, a chemical property, or a biological property 205 of the process (or manufacturing) to be developed. For example, when mechanistic model 206 is used to develop a nucleic acid therapy, data processing system 203 may access mechanistic model 206 based on a chemical property of nucleic acid molecules.

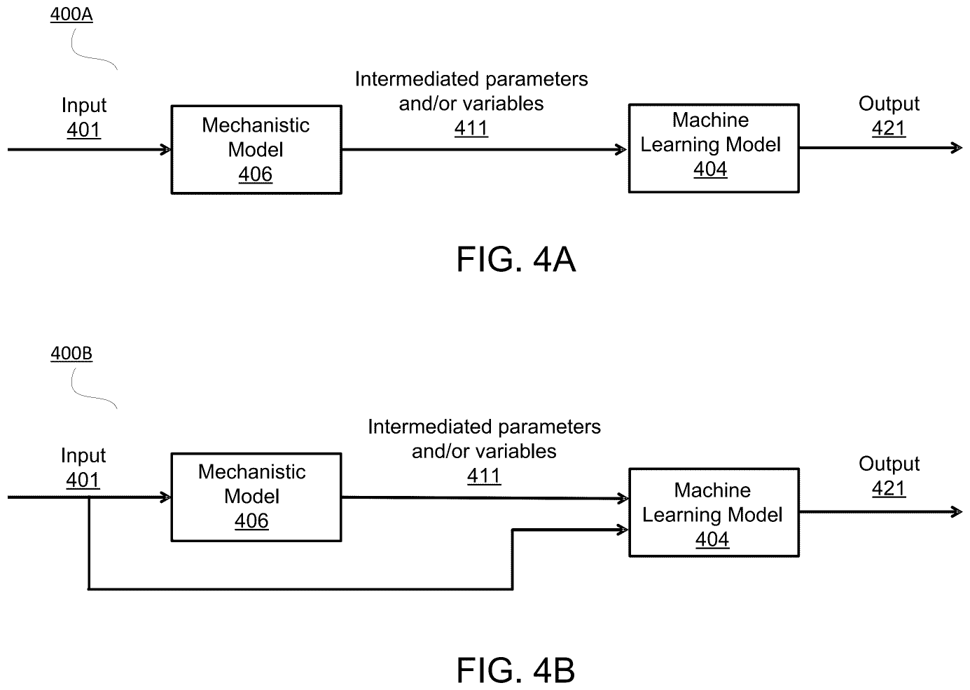

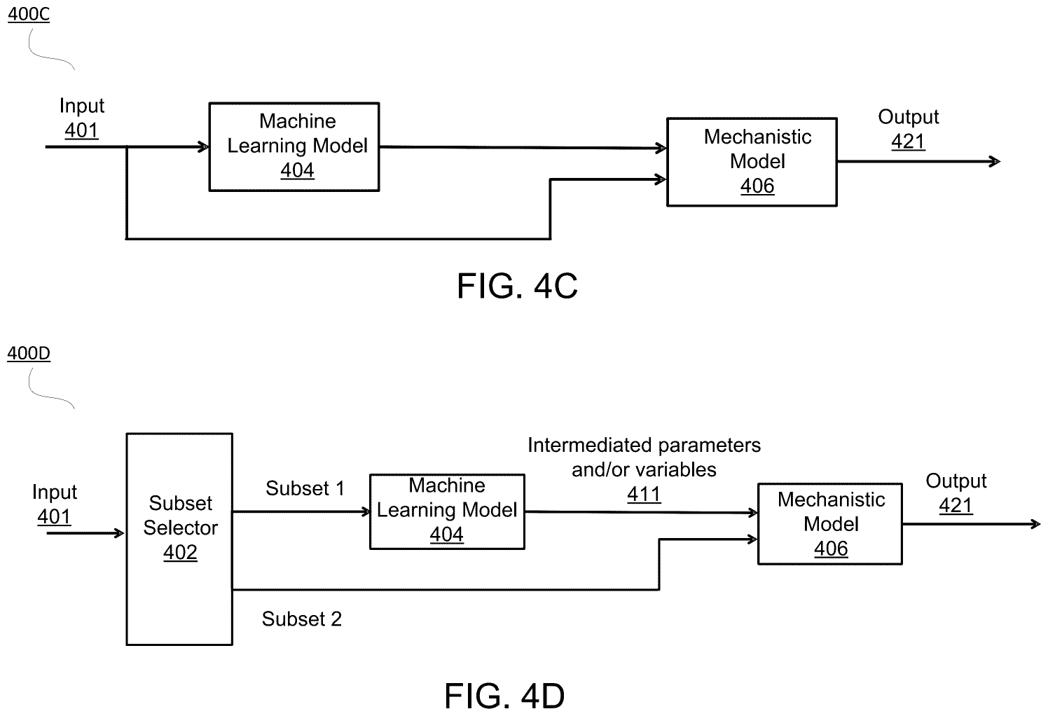

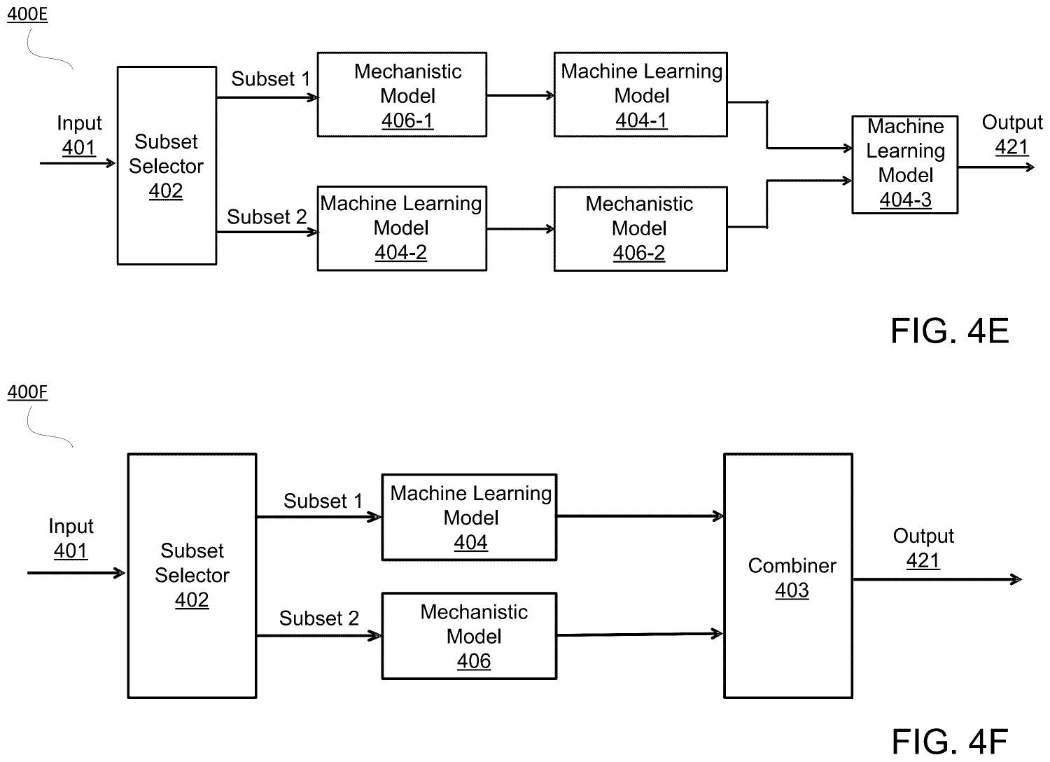

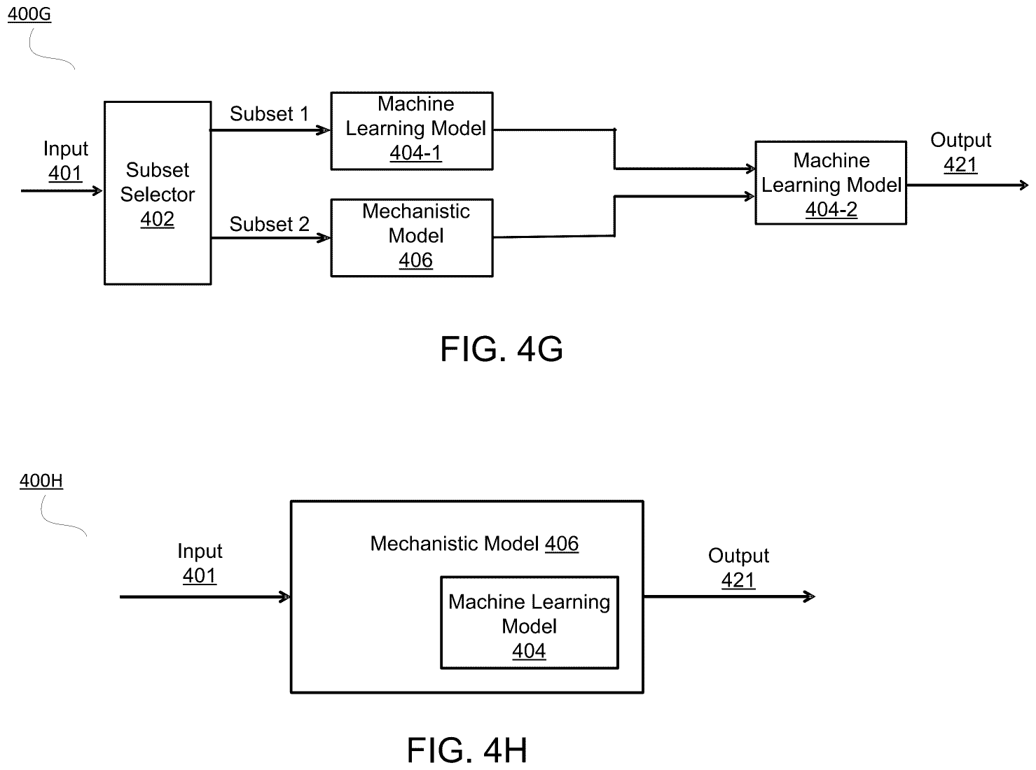

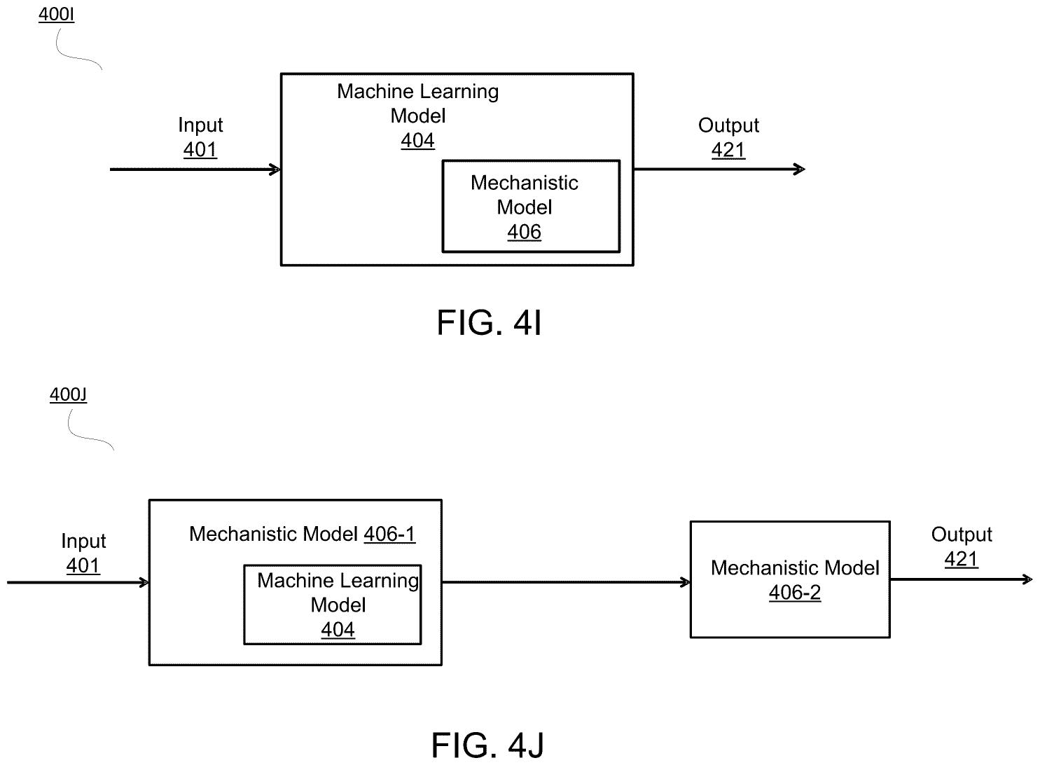

With machine learning model 204 and mechanistic model 206 selected/accessed, data processing system 203 integrates the two at operation 207 to create an integrated model, also referred to as hybrid model 208. The integration operation 207 is described later in detail with reference to FIGS. 4A-4J. As described herein, a data structure stores data representing a model (e.g., model 204 and/or model 206). This data structure is stored in memory and the data processing system 203 applies the values of fields in the data structure to input data, e.g., to produce output. That is, the data structure includes fields that store values or other data representing the model itself. In some examples, data processing system 203 includes a parser to parse input data, e.g., to identify a structure of the data, to identify fields and so forth. From the parsed data, data processing system 203 identifies—from the structure of the data—fields and values that are input into the models and/or applied to the models, using the techniques described here.

Integrated model 208 can be applied to input data 201 to make predictions in the process development or manufacturing for CGT. Because integrated model 208 has both a machine learning component and a mechanistic component, in some implementations data processing system 203 can select whether to make predictions using (i) the machine learning component only, (ii) the mechanistic component only, or (ii) the integration of machine learning and mechanistic components. The selection can be based on, e.g., the nature and the degree of uncertainty of data 201.

In some implementations, one or more values of one or more parameters of integrated model 208 are adjusted to reduce prediction uncertainty. For example, after applying integrated model 208 to data 201 for CGT development, data processing system 203 evaluates the prediction results based on actual outcomes of the CGT development. Data processing system 203 thus determines to modify the values of one or more parameters of integrated model 208 according to the actual outcomes, resulting in adjusted integrated model 208′. The adjustment can be iteratively done for a limited number of times, or can be done automatically done as the process development or manufacturing progress.

Example parameters of integrated model 208 include prefactors in biochemical rate expressions, prefactors in cellular uptake rates, time scales for transport of molecules between nucleus and organelles, and molecular diffusivities. Example methods of the adjustment include Kalman filtering, parameter-adaptive Luenberger observers, and Bayesian adaptive ensemble Kalman filtering. Integrated model 208 and adjusted integrated model 208′, which are used in making the predictions, may be collectively referred to as predictive models as described in FIG. 1.

As described above with reference to FIGS. 1 and 2, implementations of the disclosure combine features of machine learning and mechanistic models to reduce uncertainty in CGT development. The implementations do not require complex training data sets but considers the properties of the process/manufacturing. As such, the implementations can be used in a broad range of CGT development applications, with a scale possibly as small as 1 mL per production run to as large as 25,000 L per production run.

FIG. 3A provides an example chart illustrating the selection 300 of one or more machine learning models, according to some implementations. Consistent with the above description with reference to FIG. 2, the selection can be done by a data processing system based on one or more attributes of input data. FIG. 3A illustrates three example attributes, namely, nonlinearity, collinearity, and dynamics. FIG. 3A also illustrates a number of candidate machine learning models available for selection. These machine learning models include: algebraic learning via elastic net (ALVEN); canonical variate analysis (CVA); dynamic ALVEN (DALVEN); elastic net; multivariable output error state space (MOESP); partial least squares (PLS); random forest (RF); recurrent neural network (RNN); ridge regression (RR); spare PLS; state-space autoregression exogeneous (SSARX); and kernel support vector regression (kSVR).

In selection 300, data are characterized based on three attributes: nonlinearity; collinearity; and dynamics. Specifically, by accessing each data item, a data processing system can determine whether the data items sufficiently demonstrate an attribute of nonlinearity, an attribute of collinearity, or an attribute of dynamics. The data may demonstrate one, two, or all of the three attributes. In the triangular chart of FIG. 3A, each vertex corresponds to data that demonstrate one attribute, each edge corresponds to data that demonstrate two attributes at the same time, and the center of the triangle corresponds to data that demonstrate all three attributes at the same time. Next to the vertex/edge/center are examples of machine learning models that can be used for the corresponding data. As an example, data that demonstrate only collinearity correspond to the lower left vertex of the triangle. According to selection 300, machine learning models based on one or more of PLS, sparse PLS, RR, or elastic net can be selected to handle this type of data. As another example, data that demonstrate both collinearity and dynamics correspond to the bottom edge of the triangle. According to selection 300, machine learning models based on one or more of CVA, MOSEP, or SSARX can be selected to handle this type of data. Description of using these machine learning models for general data analytics can be found in Sun and Braatz, “Smart process analytics for predictive modeling,” Computers & Chemical Engineering, vol. 144, 107134, Jan. 4, 2021, which is incorporated by reference in this application. Besides the three example attributes, other attributes, such as non-normality, can also be used in the selection of machine learning models in some implementations.

FIG. 3B illustrates an example biomanufacturing process 350 in which modelling is applied, according to some implementations. In FIG. 3B, input parameters define the operation of the biomanufacturing process 350 in which bioreactions relating to the production of cells and/or its products occur. These input parameters, such as dissolved oxygen (DO), pH value, and impeller speed (measured in revolutions per minute [RPM]), affect the physical, chemical, and/or biological phenomena in the biomanufacturing process 350. By applying mathematical expressions that are functions of the input parameters, a model (e.g., a mechanistic model) may predict internal cell states and/or process performance, e.g., the production rate and quality of the cells and/or products produced by the bioreactor. Examples of internal cell states include the concentrations of species in the cells and/or its subcellular structures.

With one or more machine learning models selected based on data attributes and one or more mechanistic models accessed based on process or manufacturing properties, the two types of models are integrated to create an integrated model. A number of mechanisms to integrate the two types of models are available, with several examples described below with reference to FIGS. 4A-4J. In the below description, the term “models” means “one or more models.” Likewise, the terms “variables” and “parameters” mean “one or more variables” and “one or more parameters,” respectively.

FIG. 4A illustrates an example mechanism 400A for integrating one or more machine learning models 404 with one or more mechanistic models 406, according to some implementations. In 400A, machine learning models 404 and mechanistic models 406 are arranged in series, forming one data route of two stages. Input 401, which can be data 101 of FIG. 1 or data 201 of FIG. 2, is first input to mechanistic models 406. Based on a physical property, a chemical property, or a biological property involved in the process development or manufacturing, mechanistic models 406 make a prediction from input 401 and outputs one or more intermediate parameters or variables 411. Intermediate parameters or variables 411 are then fed to machine learning models 404, which output final prediction 421 of the integration. In this example, because intermediate parameters or variables 411 are results of a mechanistic prediction that follows the physical/chemical/biological property, the subsequent machine learning prediction based on intermediate parameters or variables 411 can be more relevant to the actual development or manufacturing than machine learning predictions based purely on raw input data 401.

FIG. 4B illustrates an example mechanism 400B for integrating one or more machine learning models with one or more mechanistic models, according to some implementations. Similar to 400A, machine learning models 404 and mechanistic models 406 are arranged in series in 400B. Input 401 has two possible data routes: a two-stage route having both mechanistic models 406 and machine learning models 404; and a single-stage route having machine learning models 404 only. Thus, in addition to receiving intermediate parameters or variables 411 for making a machine learning prediction, machine learning models 404 directly receives input 401 as another source of input. Machine learning models 404 can use the two sources of input as, e.g., references for each other to potentially filter out outliers or mistakes in intermediate parameters or variables 411. This mechanism can thus increase prediction accuracy.

FIG. 4C illustrates an example mechanism 400C for integrating one or more machine learning models with one or more mechanistic models, according to some implementations. Similar to 400A, machine learning models 404 and mechanistic models 406 are arranged in series in 400C, forming one data route of two stages. Different from the arrangement in 400A, machine learning models 404 are arranged first in 400C to receive input 401 and output intermediate parameters or variables 411, and mechanistic models 406 output final prediction 421 of the integration based on intermediate parameters or variables 411. In this example, the prediction of mechanistic models 406 can be regarded as a refinement of the machine learning prediction based on the physical/chemical/biological property involved in the process development or manufacturing.

FIG. 4D illustrates an example mechanism 400D for integrating one or more machine learning models with one or more mechanistic models, according to some implementations. In this example, subset selector 402 is introduced to divide data items in input 401 into two subsets 1 and 2, which may or may not overlap. Subset 1 is fed to a two-stage data route similar to mechanism 400C of FIG. 4C. Subset 2 is fed to a single-stage data route directly as input to mechanistic models 406.

Keeping with FIG. 4D, the selection of the data route for feeding a data item can be based on various factors. As an example, if subset selector 402 determines that machine learning models 404 are well-trained to handle the type of a particular data item, then subset selector 402 can send the particular data item to subset 1 to be processed first by machine learning models 404 and then by mechanistic models 406. Otherwise, subset selector 402 can send the particular data item to subset 2 to be processed directly by mechanistic models 406. As another example, if subset selector 402 determines that machine learning models 404 have limited computing capacity and would become a bottleneck of the flow, then subset selector 402 can, upon its own discretion or according to some predetermined rules, send some data items to subset 2 to bypass machine learning models 404, thereby reducing computation latency.

FIG. 4E illustrates an example mechanism 400E for integrating one or more machine learning models with one or more mechanistic models, according to some implementations. Similar to 400D of FIG. 4D, 400E uses subset selector 402 to divide input 401 into subsets 1 and 2, each going through a two-stage data route in parallel. Subsets 1 and 2 may or may not overlap. Subset 1 first goes through mechanistic models 406-1 and then machine learning models 404-1, while subset 2 first goes through machine learning models 404-2 and then mechanistic models 406-2. Predictions from the two subsets are then both input to machine learning models 404-3 as a third stage, for a prediction of the integration. In 400E, the two instances of mechanistic models 406-1 and 406-2 may or may not be the same, and the three instances of machine learning models 404-1 to 404-3 may or may not be the same. By branching out input 401 into multiple routes, mechanism 400E can potentially improve computation speed. Furthermore, by having three stages of prediction, mechanism 400E can potentially increase prediction accuracy compared with two-stage mechanisms and single-stage mechanism.

FIG. 4F illustrates an example mechanism 400F for integrating one or more machine learning models with one or more mechanistic models, according to some implementations. In 400F, subsets 1 and 2 each go through a single-stage route having machine learning models 404 and mechanistic models 406, respectively. The outputs of the two parallel routes are then combined by combiner 403 as output 421 of the integration.

FIG. 4G illustrates an example mechanism 400G for integrating one or more machine learning models with one or more mechanistic models, according to some implementations. Different from 400F, 400G replaces combiner 403 with machine learning models 404-2 as a second stage prediction. The prediction made by machine learning models 404-2 forms output 421 of the integration.

FIG. 4H illustrates an example mechanism 400H for integrating one or more machine learning models with one or more mechanistic models, according to some implementations. In 400H, machine learning models 404 are embedded within mechanistic models 406 to assist mechanistic models 406 in the prediction. For example, built-in machine learning models 404 can assist mechanistic models 406 in data acquisition and/or classification to accelerate the mechanistic prediction process.

FIG. 4I illustrates an example mechanism 400I for integrating one or more machine learning models with one or more mechanistic models, according to some implementations. In 400I, mechanistic models 406 are embedded within machine learning models 404. Specifically, machine learning models 404 have built-in constraints imposed according to a physical/chemical/biological property of mechanistic models 406. With this mechanism, output 421, which is a machine learning prediction, leverages information from mechanistic models 406. As such, output 421 describes the actual process more closely than a pure machine learning model.

FIG. 4J illustrates an example mechanism 400J for integrating one or more machine learning models with one or more mechanistic models, according to some implementations. In addition to embedding machine learning models 404 within mechanistic models 406-1, 400J introduces mechanistic models 406-2 for a stage two prediction. The addition of mechanistic models 406 can potentially improve prediction accuracy.

In the integration mechanisms described above, input data can be structured in a variety of ways. As an example, each data item can be structured as a one-dimension or multi-dimension vector with each element corresponding to an aspect or a feature of the process development or manufacturing. As another example, each data item can be structured as a tree with the root corresponding to a main aspect or feature and each branch underneath corresponding to a sub-aspect or sub-feature. Many other example data structures are available. One of ordinary skill in the art, after reading this disclosure, would be able to implement the integration mechanisms described above using one or more data structures suitable for the process development or manufacturing.

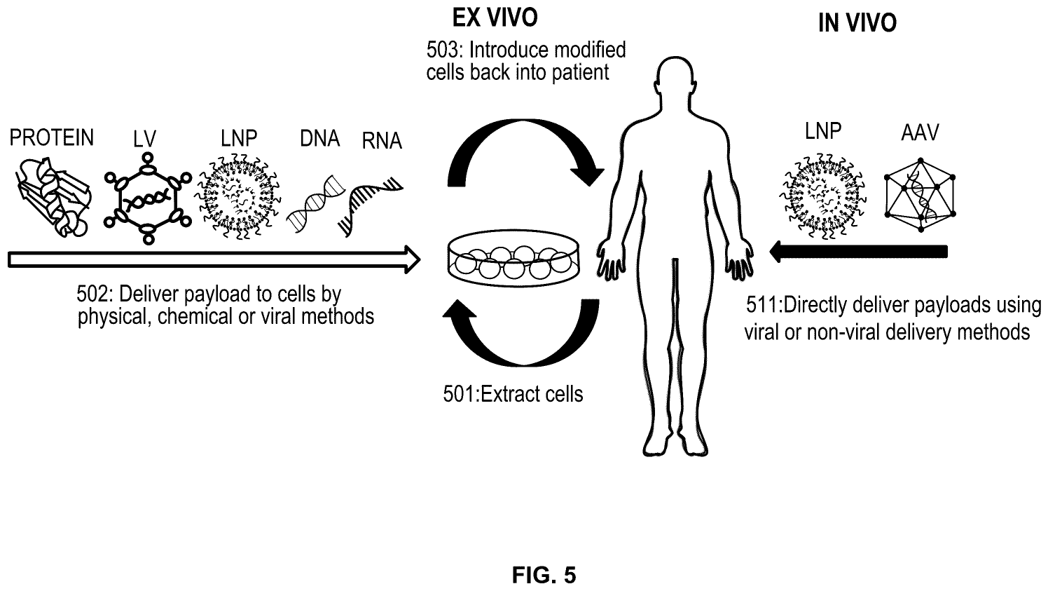

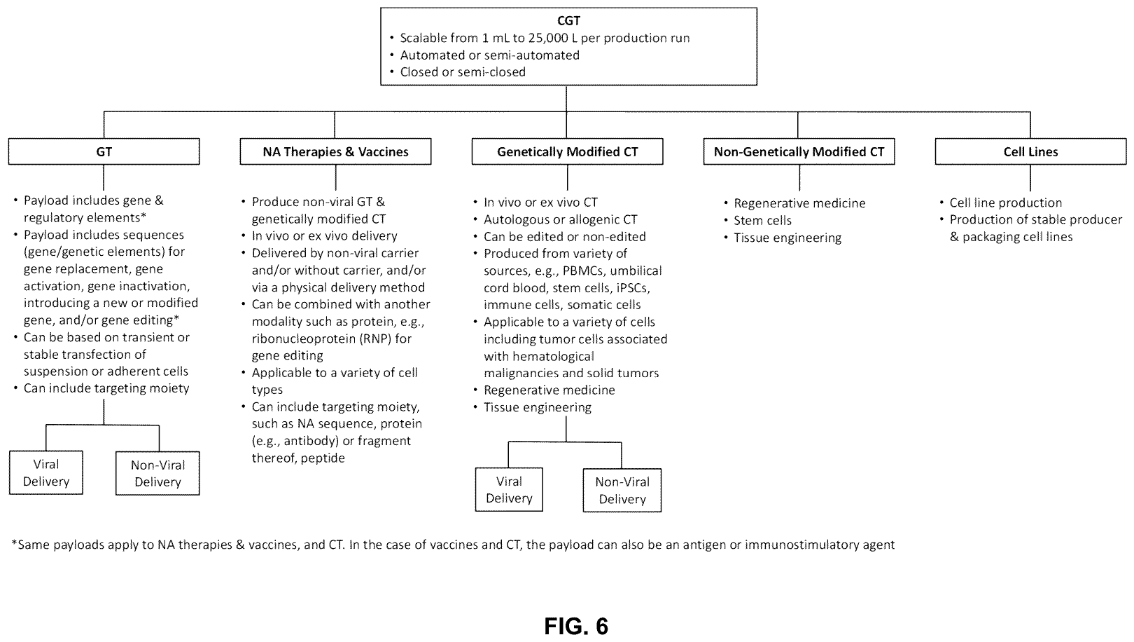

After integration, the integrated model can be used in a wide variety of applications for making predictions and reducing uncertainties in the process development and/or manufacturing for CGT. Example modalities and techniques of CGT are described below, with reference to FIGS. 5 and 6.

FIG. 5 illustrates example CGT modalities, according to some implementations. In general, CGT includes CT and GT. CT involves ex vivo or in vivo production, and may or may not include genetic modification. GT involves directly delivering therapeutic genetic elements to a human subject. GT can be achieved using a delivery vehicle such as a viral vector, a bacterial vector, a non-viral carrier, and/or a physical method, or can be delivered without a delivery vehicle. Said genetic element can encode, for example, a full-length protein, a protein fragment, a polypeptide, a peptide, a regulatory element, or a transposable element. Examples of encoded proteins and fragments include enzymes, structural proteins, regulatory proteins, antibodies, cytokines, antigens, and transcription factors. Examples of regulatory elements include promoters, enhancers, genetic switches, and logic gates. Examples of transposable elements include transposons, such as Sleeping Beauty, piggyBac, and Tol2. Examples of genetic switches include on switches, off switches, on-off switches, and dimmer switches.

Ex vivo implementations of CT include steps 501-503. At 501, cells, such as stem cells or non-stem cells, are extracted from the body of a human, who may or may not be the human recipient, or from a non-human animal. At 502, the extracted cells may be manipulated, modified, and/or amplified. In addition, the cells may be genetically modified with payloads delivered via a viral vector, a bacterial vector, a non-viral carrier, and/or by physical means. Example of payloads include deoxyribonucleic acid (DNA), ribonucleic acid (RNA), non-natural NA, peptide, and/or protein. At step 503, the resultant cell therapy product can be unicellular or multicellular, and is transferred to the body of the human subject for therapeutic or preventive purposes.

In vivo implementations of CT include step 511 in which payloads are directly delivered to the body of the human subject. Said payload may be delivered via a viral vector, a bacterial vector, a non-viral carrier, and/or by physical means. Example of payloads include deoxyribonucleic acid (DNA), ribonucleic acid (RNA), non-natural nucleic acid, peptide, and/or protein. Step 511 may be similarly used in GT implementations, which are also in vivo, to deliver genetic sequences to the body of the human subject.

The integrated model can be applied to any of steps 501-503 and 511, as well as numerous other techniques involved in CGT. Examples of these techniques are described below with reference to FIG. 6.

FIG. 6 illustrates example techniques involved in CGT, according to some implementations. These techniques are broadly classified into four categories: GT, NA, genetically modified CT, and non-genetically modified CT. The scale of production in these techniques can range from 1 mL per production run to 25,000 L per production run, and the mode of production can be, e.g., batch, fed-batch, perfusion, continuous, semi-continuous, or hybrid of fed-batch and perfusion.

In GT, a payload can include one or more genes and/or regulatory sequences, and can include one or more non-coding sequences. The payload can be used for, e.g., gene replacement, gene activation, gene inactivation, introducing a new or modified gene, cell reprogramming, transdifferentiation, and/or gene editing. The payload can be delivered via a viral vector, a non-viral vector such as bacteria, a non-viral carrier, and/or via a physical means of delivery. The GT can include at least one targeting moiety, which may be combined with the payload or may be intrinsic to the payload. Targeting moieties can include an NA sequence, a protein (e.g., antibody), a protein fragment (e.g., antibody fragment), a peptide, a monosaccharide, a polysaccharide, an aptamer, a dendrimer, a small molecule, or a centyrin. Said moieties can be combined with, fused to, conjugated with, or attached to the vector or payload.

The GT can involve performing transient transfection, stable transfection, or transduction of suspension or adherent cells to produce the therapeutic substance, such as a viral vector that carries a gene of interest. The GT can involve performing transfection of a stable producer or packaging cell line grown in suspension or grown as adherent cells to produce the therapeutic substance. Examples of cell lines include HEK293 and variants thereof (e.g., HEK293T), Sf9, HeLa, A469, CAP, AGELHN, PER.C6, NS01, COS-7, BHK, CHO, VERO, MDCK, BRL3A, HepG2, primary human cells, peripheral blood mononuclear cells (PBMC), immune cells, T-cells, human stem cells, induced pluripotent stem cells, or somatic cells. In addition, the GT can involve producing the therapeutic substance in a transfection-free system, such as a self-attenuating adenovirus-based system (e.g., a system based on Tetracycline-Enabled Self-Silencing Adenovirus [TESSA]) for viral vector production, or an oncolytic virus that selectively replicates in and kills the target cells. Examples of viral vectors include Adeno-associated virus (AAV), Lentivirus (LV), Adenovirus (Ad), Baculovirus, Herpes Simplex Virus (HSV), Retrovirus, Oncolytic virus, Parvovirus, Annellovirus, and Bacteriophage.

All of the above GT techniques can use the integrated model to reduce uncertainties. For example, the integrated model can be used in the generation of viral vectors or non-viral carriers, the generation and delivery of payloads, the performance of transient transfection, etc.

NA therapies and vaccines can involve producing and delivering an NA-based therapy or vaccine encoding a therapeutic and/or protective moiety. Delivery can be in vivo or ex vivo. NA therapies and vaccines can be applied to a variety of cell types, with or without a specific target. Such cell types include immune cells, tumor cells, cardiac cells, ocular cells, retinal cells, lung cells, skin cells, muscle cells, liver cells, pancreatic cells, intestinal cells, brain cells, and neurological cells, to name a few.

The NA therapy or vaccine can include DNA, plasmid DNA (pDNA) (including bacmids, nanoplasmids, linearized pDNA, etc.), RNA, messenger RNA (mRNA), small activating RNA (saRNA), small interfering RNA (also known as short interfering RNA, silencing RNA, or siRNA), microRNA (miRNA), circular RNA, antisense oligonucleotide (ASO), doggybone DNA (dbDNA), minicircle DNA (mcDNA), minimalistic immunologically defined gene expression (MIDGE), closed-ended DNA (ceDNA), synthetic DNA, or a non-natural NA, including nucleotides or nucleosides, which can be non-natural or modified, and peptides, including non-natural chemistries and multidimensional structures.

The NA therapy or vaccine can include one or more non-identical NA molecules, for example where each encodes a different sequence. It can also include one or more non-NA elements. For example, the NA therapy can include a protein or protein fragment, such as a ribonucleoprotein (RNP), for gene editing. It can also include one or more targeting moieties, such as to enhance delivery to a specific organ, tissue, cell type, or subcellular compartment. The one or more targeting moieties can include an NA sequence, a protein (e.g., antibody), a protein fragment (e.g., antibody fragment), a peptide, a monosaccharide, a polysaccharide, an aptamer, a dendrimer, a small molecule, or a centyrin. The moieties can be ligands that are combined with, fused to, conjugated with, or attached to the NA payload. The moieties may also be encoded or intrinsic to the payload itself.

NA therapies and vaccines can further involve producing an NA, modifying the NA chemically or enzymatically, and delivering the NA either by combining the NA with a non-viral carrier and/or via a physical delivery method. Examples of non-viral carriers include lipid nanoparticles (LNPs), solid lipid nanoparticles (SLNs), nanostructured lipid carriers (NLCs), liposomes, lipoplexes, polymeric nanoparticles, lipid-polymer hybrid nanoparticles, inorganic nanoparticles, exosomes, virus-like particles, extracellular vesicles, cell-penetrating peptides, cationic polymers (e.g., PEI, PLA, PLGA, chitosan), dendrimers, aptamers, and centyrins. Examples of physical delivery methods include electroporation, cell squeezing, needles (including micro- and nano-needles), patches, iontophoresis, biolistic delivery (including gene gun and particle bombardment), sonoporation, ultrasound-mediated microbubbles, hydroporation, photoporation, and magnetofection.

All of the above NA-based therapy and vaccine techniques can use the integrated model to reduce uncertainties. For example, the integrated model can be used in producing, modifying, and delivering the NA, in causing the production of the NA sequences (e.g., either produced together in the same reaction or produced in separate reactions and then mixed in a single product), in applying the therapies and vaccines to cells of various types, in generating the non-viral carriers, and in conducting the physical delivery method.

A CT can be created by, e.g., transduction with a viral vector or transfection with an NA. In CT, the resultant cells can be genetically modified or non-genetically modified, stem cell-based or non-stem cell based, and unicellular or multicellular. In addition, the resultant cells can be autologous (patient-specific). Autologous CT involves obtaining cells from a source (e.g., stem cells, human pluripotent stem cells, including induced pluripotent stem cells and embryonic stem cells, non-stem cells, or cell lines, derived from a variety of sources, such as peripheral blood, bone marrow, umbilical cord blood, placenta, skin, eye, muscle, and tumor) from a human subject, culturing and expanding the cells outside of the body (ex vivo), and reintroducing the resulting CT product into the same subject. The process can include enrichment for one or more specific cell types or phenotypes. The process can include genetic modification. In addition, the process can include gene editing to produce one or more gene edits.

The cells generated can be allogeneic (used to treat multiple patients). Allogeneic CT involves obtaining cells from a source (e.g., stem cells, human pluripotent stems cells, including induced pluripotent stem cells and embryonic stem cells, non-stem cells, or cell lines) derived from a variety of sources, such as human peripheral blood from a healthy donor, umbilical cord blood, placenta, and skin), and creating a master cell bank (MCB), which is used as the source to create a cell population that is processed according to the demands of the specific therapy. The final cell populations are then used to treat one or more patients. The process can include enrichment for one or more specific cell types or phenotypes. The process can include genetic modification. In addition, the process can include gene editing to produce one or more gene edits.

Genetically modified CT can involve modifying particular genes and/or regulatory sequences within the cells. Genetically modified CT can be applied to a variety of cell types, including immune cells, tumor cells, cardiac cells, ocular cells, retinal cells, lung cells, skin cells, pancreatic cells, intestinal cells, muscle cells, liver cells, brain cells, and neurological cells, to name a few.

Genetically modified CT can be applied to tumor cells associated with hematological malignancies and solid tumors. For example, CT can involve production of a genetically modified chimeric antigen receptor T-cell (CAR T-cell), a gamma delta T-cell, a natural killer (NK) cell, an engineered T-cell receptor (TCR), a tumor-infiltrating lymphocyte (TIL), a macrophage, a dendritic cell, a hematopoietic stem cell (HSC), or a mesenchymal stem/stromal cell (MSC).

Genetically modified CT can include one or more targeting moieties. The one or more targeting moieties can include an NA sequence, a protein (e.g., antibody), a protein fragment (e.g., antibody fragment), a peptide, a monosaccharide, a polysaccharide, an aptamer, a dendrimer, a small molecule, or a centyrin. Said moieties can be combined with, fused to, conjugated with, attached to the cell. The moiety may also be encoded or intrinsic to the cell itself, and may be expressed on the cell surface. In genetically modified CT, cells can be generated and modified ex vivo or in vivo.

Non-genetically modified CT can involve regenerative medicine, stem cell therapy, or tissue engineering. Non-genetically modified CT can be applied to a variety of cells including immune cells, tumor cells, cardiac cells, ocular cells, retinal cells, lung cells, pancreatic cells, intestinal cells, muscle cells, skin cells, bone cells, liver cells, brain cells, and neurological cells, to name a few.

All of the above genetically modified and non-genetically modified CT techniques can use the integrated model to reduce uncertainties. For example, the integrated model can be used in generating autologous or allogeneic cells, editing the cells, and producing viral or non-viral delivery means.

In addition to the techniques described in each classification, the integrated model can be applied to CGT techniques for producing cell lines. For example, using input data obtained from a cell line or cell population of a first type, the integrated model can be applied to producing a cell line or cell population of a second type different from the first type. The two cell lines/populations can have heterogeneous cell populations or clonal cell populations, where heterogeneous cell populations can have intracellular heterogeneity or cell surface heterogeneity. The production can be automated or semi-automated and can be a semi-closed system or a closed system.

In some implementations, the integrated model can be applied to production of a stable cell line or packaging of a cell line. In some implementations, the integrated model can be applied to performance of transfection (e.g., transient transfection) or transduction of one or more stable producer host cell lines or one or more packaging host cell lines, which can be used in, e.g., GT.

The techniques described above are only a few of numerous examples to which the integrated model can apply to reduce uncertainties. Thanks to their high scalability, customizability, and prediction accuracy, implementations of the present disclosure can be used in numerous applications of process development and manufacturing of biopharmaceuticals.

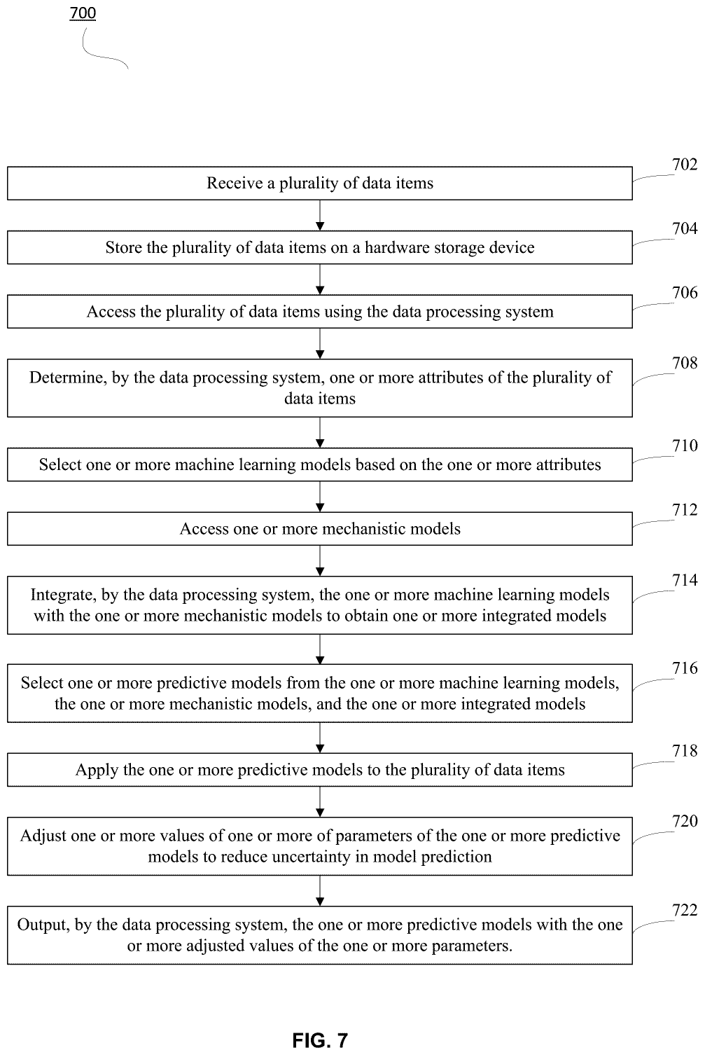

FIG. 7 illustrates a flowchart of an example method 700, according to some implementations. Method 700 can be implemented as software code on a computer. One or more steps of method 700 may correspond to the steps or operations described with reference to FIGS. 1 and 2.

At 702, method 700 involves receiving a plurality of data items, such as data 101 in FIG. 1 or data 201 in FIG. 2.

At 704, method 700 involves storing the plurality of data items on a hardware storage device, such as storage 202 in FIG. 2.

At 706, method 700 involves accessing the plurality of data items using the data processing system, such as data processing system 203 in FIG. 2.

At 708, method 700 involves determining, by the data processing system, one or more attributes of the plurality of data items. These attributes can include those described with reference to FIG. 3A.

At 710, method 700 involves selecting one or more machine learning models based on the one or more attributes. The one or more machine learning models can include those described with reference to FIG. 3A.

At 712, method 700 involves accessing one or more mechanistic models. Consistent with the description above, accessing one or more mechanistic models can be based on one or more physical/chemical/biological properties involved in the process development or manufacturing.

At 714, method 700 involves integrating, by the data processing system, the one or more machine learning models with the one or more mechanistic models to obtain one or more integrated models. The integration can correspond to step 207 of FIG. 2, and can use one or more mechanisms described with reference to FIGS. 4A-4J.

At 716, method 700 involves selecting one or more predictive models from the one or more machine learning models, the one or more mechanistic models, and the one or more integrated models. The selection can be based on the input data items, the nature of uncertainties, and the available computing resources. Depending on the selection, either the one or more machine learning models alone, the one or more mechanistic models alone, or the integrated of the two may be selected as the one or more predictive models for reducing uncertainties.

At 718, method 700 involves applying the one or more predictive models to the plurality of data items. The application can be used in the CGT techniques described with reference to FIGS. 5 and 6.

At 720, method 700 involves adjusting one or more values of one or more parameters of the one or more predictive models to reduce uncertainty in model prediction. The one or more parameters and the adjustment thereof can be similar to the same described with reference to FIG. 2.

At 722, method 700 involves outputting, by the data processing system, the one or more predictive models with the one or more adjusted values of the one or more parameters. The output one or more predictive models, with parameters adjusted, can be similar to adjusted integrated model 208′.

As described above, with the features described herein, the integration of a mechanistic model with a machine learning model advantageously improves the capability and efficiency of prediction in process development and manufacturing of biopharmaceuticals, resulting in significant increase in scalability and reduction of cost.

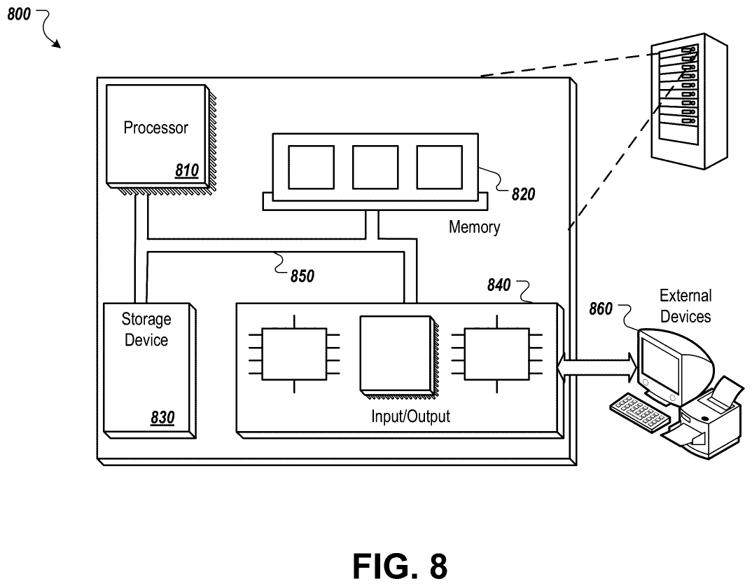

FIG. 8 is a block diagram of an example computer system 800 in accordance with embodiments of the present disclosure. Storage 202 and data processing system 203, for example, can be implemented as components of the computer system 800. The system 800 includes a processor 810, a memory 820, a storage device 830, and one or more input/output interface devices 840. Each of the components 810, 820, 830, and 840 can be interconnected, for example, using a system bus 850.

The processor 810 is capable of processing instructions for execution within the system 800. The term “execution” as used here refers to a technique in which program code causes a processor to carry out one or more processor instructions. In some implementations, the processor 810 is a single-threaded processor. In some implementations, the processor 810 is a multi-threaded processor. The processor 810 is capable of processing instructions stored in the memory 820 or on the storage device 830. The processor 810 may execute operations such as those described with reference to other figures described herein.

The memory 820 stores information within the system 800. In some implementations, the memory 820 is a computer-readable medium. In some implementations, the memory 820 is a volatile memory unit. In some implementations, the memory 820 is a non-volatile memory unit.

The storage device 830 is capable of providing mass storage for the system 800. In some implementations, the storage device 830 is a non-transitory computer-readable medium. In various different implementations, the storage device 830 can include, for example, a hard disk device, an optical disk device, a solid-state drive, a flash drive, magnetic tape, or some other large capacity storage device. In some implementations, the storage device 830 may be a cloud storage device, e.g., a logical storage device including one or more physical storage devices distributed on a network and accessed using a network. In some examples, the storage device may store long-term data. The input/output interface devices 840 provide input/output operations for the system 800. In some implementations, the input/output interface devices 840 can include one or more network interface devices, e.g., an Ethernet interface, a serial communication device, e.g., an RS-232 interface, and/or a wireless interface device, e.g., an 802.11 interface, a 3G wireless modem, a 4G wireless modem, a 5G wireless modem, etc. A network interface device allows the system 800 to communicate, for example, transmit and receive data. In some implementations, the input/output device can include driver devices configured to receive input data and send output data to other input/output devices, e.g., keyboard, printer and display devices 860. In some implementations, mobile computing devices, mobile communication devices, and other devices can be used.

A server can be distributively implemented over a network, such as a server farm, or a set of widely distributed servers or can be implemented in a single virtual device that includes multiple distributed devices that operate in coordination with one another. For example, one of the devices can control the other devices, or the devices may operate under a set of coordinated rules or protocols, or the devices may be coordinated in another fashion. The coordinated operation of the multiple distributed devices presents the appearance of operating as a single device.

In some examples, the system 800 is contained within a single integrated circuit package. A system 800 of this kind, in which both a processor 810 and one or more other components are contained within a single integrated circuit package and/or fabricated as a single integrated circuit, is sometimes called a microcontroller. In some implementations, the integrated circuit package includes pins that correspond to input/output ports, e.g., that can be used to communicate signals to and from one or more of the input/output interface devices 840.

Although an example processing system has been described in FIG. 8, implementations of the subject matter and the functional operations described in this specification can be implemented in digital electronic circuitry, in tangibly embodied computer software or firmware, in computer hardware, including the structures disclosed in this specification and their structural equivalents, or in combinations of one or more of them. In some implementations, the computing (e.g., data processing) may occur in a central location and/or in distributed locations, e.g., involving edge computing. The computing may also involve quantum computing in some implementations.

Software implementations of the described subject matter can be implemented as one or more computer programs. Each computer program can include one or more modules of computer program instructions encoded on a tangible, non-transitory, computer-readable computer-storage medium for execution by, or to control the operation of, data processing apparatus. Alternatively, or additionally, the program instructions can be encoded in/on an artificially generated propagated signal. In an example, the signal can be a machine-generated electrical, optical, or electromagnetic signal that is generated to encode information for transmission to suitable receiver apparatus for execution by a data processing apparatus. The computer-storage medium can be a machine-readable storage device, a machine-readable storage substrate, a random or serial access memory device, or a combination of computer-storage mediums.

The terms “data processing apparatus,” “computer,” and “computing device” (or equivalent as understood by one of ordinary skill in the art) refer to data processing hardware. For example, a data processing apparatus can encompass all kinds of apparatus, devices, and machines for processing data, including by way of example, a programmable processor, a computer, or multiple processors or computers. The apparatus can also include special purpose logic circuitry including, for example, a central processing unit (CPU), a field programmable gate array (FPGA), or an application specific integrated circuit (ASIC). In some implementations, the data processing apparatus or special purpose logic circuitry (or a combination of the data processing apparatus or special purpose logic circuitry) can be hardware- or software-based (or a combination of both hardware- and software-based). The apparatus can optionally include code that creates an execution environment for computer programs, for example, code that constitutes processor firmware, a protocol stack, a database management system, an operating system, or a combination of execution environments. The present disclosure contemplates the use of data processing apparatuses with or without conventional operating systems, for example LINUX, UNIX, WINDOWS, MAC OS, ANDROID, or MS.

A computer program, which can also be referred to or described as a program, software, a software application, a module, a software module, a script, or code, can be written in any form of programming language. Programming languages can include, for example, compiled languages, interpreted languages, declarative languages, or procedural languages. Programs can be deployed in any form, including as standalone programs, modules, components, subroutines, or units for use in a computing environment. A computer program can, but need not, correspond to a file in a file system. A program can be stored in a portion of a file that holds other programs or data, for example, one or more scripts stored in a markup language document, in a single file dedicated to the program in question, or in multiple coordinated files storing one or more modules, sub programs, or portions of code. A computer program can be deployed for execution on one computer or on multiple computers that are located, for example, at one site or distributed across multiple sites that are interconnected by a communication network. While portions of the programs illustrated in the various figures may be shown as individual modules that implement the various features and functionality through various objects, methods, or processes, the programs can instead include a number of sub-modules, third-party services, components, and libraries. Conversely, the features and functionality of various components can be combined into single components as appropriate. Thresholds used to make computational determinations can be statically, dynamically, or both statically and dynamically determined.

The methods, processes, or logic flows described in this specification can be performed by one or more programmable computers executing one or more computer programs to perform functions by operating on input data and generating output. The methods, processes, or logic flows can also be performed by, and apparatus can also be implemented as, special purpose logic circuitry, for example, a CPU, an FPGA, an Arduino, or an ASIC.

Computers suitable for the execution of a computer program can be based on one or more of general and special purpose microprocessors and other kinds of CPUs. The elements of a computer are a CPU for performing or executing instructions and one or more memory devices for storing instructions and data. Generally, a CPU can receive instructions and data from (and write data to) a memory. A computer can also include, or be operatively coupled to, one or more mass storage devices for storing data. In some implementations, a computer can receive data from, and transfer data to, the mass storage devices including, for example, magnetic, magneto optical disks, or optical disks. Moreover, a computer can be embedded in another device, for example, a mobile telephone, a personal digital assistant (PDA), a mobile audio or video player, a game console, a GNSS sensor or receiver, or a portable storage device such as a universal serial bus (USB) flash drive.

The term “computer-readable medium” includes, but is not limited to, portable or non-portable storage devices, optical storage devices, and various other mediums capable of storing, containing, or carrying instruction(s) and/or data. A computer-readable medium may include a non-transitory medium in which data can be stored and that does not include carrier waves and/or transitory electronic signals propagating wirelessly or over wired connections. Examples of a non-transitory medium may include, but are not limited to, a magnetic disk or tape, optical storage media such as compact disk (CD) or digital versatile disk (DVD), flash memory, memory or memory devices. A computer-readable medium may have stored thereon code and/or machine-executable instructions that may represent a procedure, a function, a subprogram, a program, a routine, a subroutine, a module, a software package, a class, or any combination of instructions, data structures, or program statements. A code segment may be coupled to another code segment or a hardware circuit by passing and/or receiving information, data, arguments, parameters, or memory contents. Information, arguments, parameters, data, etc. may be passed, forwarded, or transmitted via any suitable means including memory sharing, message passing, token passing, network transmission, or the like.

Computer readable media (transitory or non-transitory, as appropriate) suitable for storing computer program instructions and data can include all forms of permanent/non-permanent and volatile/non-volatile memory, media, and memory devices. Computer readable media can include, for example, semiconductor memory devices such as random access memory (RAM), read only memory (ROM), phase change memory (PRAM), static random access memory (SRAM), dynamic random access memory (DRAM), erasable programmable read-only memory (EPROM), electrically erasable programmable read-only memory (EEPROM), and flash memory devices. Computer readable media can also include, for example, magnetic devices such as tape, cartridges, cassettes, and internal/removable disks. Computer-readable media can also include magneto optical disks and optical memory devices and technologies including, for example, digital video disc (DVD), CD ROM, DVD+/−R, DVD-RAM, DVD-ROM, HD-DVD, and BLURAY. The memory can store various objects or data, including caches, classes, frameworks, applications, modules, backup data, jobs, web pages, web page templates, data structures, database tables, repositories, and dynamic information. Types of objects and data stored in memory can include parameters, variables, algorithms, instructions, rules, constraints, and references. Additionally, the memory can include logs, policies, security or access data, and reporting files. The processor and the memory can be supplemented by, or incorporated in, special purpose logic circuitry.

While this specification includes many specific implementation details, these should not be construed as limitations on the scope of what may be claimed, but rather as descriptions of features that may be specific to particular implementations. Certain features that are described in this specification in the context of separate implementations can also be implemented, in combination, in a single implementation. Conversely, various features that are described in the context of a single implementation can also be implemented in multiple implementations, separately, or in any suitable sub-combination. Moreover, although previously described features may be described as acting in certain combinations and even initially claimed as such, one or more features from a claimed combination can, in some cases, be excised from the combination, and the claimed combination may be directed to a sub-combination or variation of a sub-combination.

Particular implementations of the subject matter have been described. Other implementations, alterations, and permutations of the described implementations are within the scope of the following claims as will be apparent to those skilled in the art. While operations are depicted in the drawings or claims in a particular order, this should not be understood as requiring that such operations be performed in the particular order shown or in sequential order, or that all illustrated operations be performed (some operations may be considered optional), to achieve desirable results. In certain circumstances, multitasking or parallel processing (or a combination of multitasking and parallel processing) may be advantageous and performed as deemed appropriate.

Moreover, the separation or integration of various system modules and components in the previously described implementations should not be understood as requiring such separation or integration in all implementations, and it should be understood that the described program components and systems can generally be integrated together in a single software product or packaged into multiple software products.

Accordingly, the previously described example implementations do not define or constrain the present disclosure. Other changes, substitutions, and alterations are also possible without departing from the spirit and scope of the present disclosure.

What is claimed is:

1. A method implemented by a data processing system for outputting one or more models for developing or operating a process for a cell or gene therapy (CGT), comprising:
receiving a plurality of data items;
storing the plurality of data items on a hardware storage device;
accessing the plurality of data items using the data processing system;
determining, by the data processing system, one or more attributes of the plurality of data items;
selecting one or more machine learning models based on the one or more attributes;
accessing one or more mechanistic models;
integrating, by the data processing system, the one or more machine learning models with the one or more mechanistic models to obtain one or more integrated models;
selecting one or more predictive models from the one or more machine learning models, the one or more mechanistic models, and the one or more integrated models;
applying the one or more predictive models to the plurality of data items;
adjusting one or more values of one or more parameters of the one or more predictive models to reduce uncertainty in model prediction; and
outputting, by the data processing system, the one or more predictive models with the one or more adjusted values of the one or more parameters.

2. The method of claim 1, wherein the one or more attributes comprise at least one of: nonlinearity; collinearity; nonnormality; or dynamics.

3. The method of claim 1, wherein the one or more mechanistic models are accessed based on at least one of a physical property, a chemical property, or a biological property of the process.

4. The method of claim 1, wherein integrating the one or more machine learning models with the one or more mechanistic models comprises:
arranging the one or more machine learning models and the one or more mechanistic models in a sequence comprising a first one or more models and a second one or more models;
transmitting an output of the first one or more models to the second one or more models;
transmitting data to the second one or more models; and
obtaining an output of the second one or more models.

5. The method of claim 1, wherein integrating the one or more machine learning models with the one or more mechanistic models comprises:
determining a first one or more models and a second one or more models from the one or more machine learning models and the one or more mechanistic models;
transmitting input data to the first one or more models;
constraining a prediction of the first one or more models using the second one or more models; and
obtaining an output of the first one or more models.

6. The method of claim 1,
wherein the plurality of data items is obtained from a cell population of a first type,
wherein the method further comprises:
causing production of a cell population of a second type using one or more output models, and
wherein the second type is different from the first type.

7. The method of claim 6, wherein each of the cell population of the first type and the cell population of the second type comprises at least one of: heterogeneous cell populations; or clonal cell populations.

8. The method of claim 7, wherein the heterogeneous cell populations have at least one of: intracellular heterogeneity; or cell surface heterogeneity.

9. The method of claim 1, further comprising:
causing production of a stable cell line using one or more output models.

10. The method of claim 9, wherein the stable cell line comprises at least one of:
HEK293 cells; HEK293T cells; Sf9 cells; HeLa cells, A469 cells; CAP cells; AGELHN cells; Per.C6 cells; NS01 cells; COS-7 cells; BHK cells; CHO cells; VERO cells; MDCK cells; BRL3A cells; HepG2 cells; primary human cells; peripheral blood mononuclear cells (PBMC); immune cells, T-cells; human stem cells; induced pluripotent stem cells; or somatic cells.

11. The method of claim 1, wherein a scale of the CGT is within a range of 1 mL per production run to 25,000 L per production run.

12. The method of claim 1, wherein the CGT uses one or more output models in cells grown for at least one mode of:
batch; fed-batch; perfusion; continuous; semi-continuous; or hybrid of fed-batch and perfusion.

13. The method of claim 1, wherein the CGT uses one or more output models to cause an automated or semi-automated production.

14. The method of claim 13, wherein the production is in a closed or semi-closed system.

15. The method of claim 1, wherein the CGT comprises a gene therapy.

16. The method of claim 15, wherein the gene therapy comprises using one or more payloads for at least one of:
gene replacement; gene activation; gene inactivation; introducing a new or modified gene; or gene editing.

17. The method of claim 15, further comprising:
causing generation of one or more viral vectors for the gene therapy using one or more output models.

18. The method of claim 17, where the viral vector comprises at least one of:
Adeno-associated virus; Lentivirus; Adenovirus; Baculovirus; Herpes Simplex Virus; Retrovirus; Oncolytic virus; Parvovirus; Annellovirus; or a Bacteriophage.

19. The method of claim 15, further comprising:
causing performance of transient transfection, stable transfection, or transduction for the gene therapy using one or more output models.

20. The method of claim 18, further comprising: causing performance of transient transfection, stable transfection, or transduction of suspension or adherent cells.

21. The method of claim 20, wherein the suspension or adherent cells comprise at least one of:
HEK293 cells; HEK293T cells; Sf9 cells; HeLa cells, A469 cells; CAP cells, AGELHN cells; Per.C6 cells; NS01 cells; COS-7 cells; BHK cells; CHO cells; VERO cells; MDCK cells; BRL3A cells; HepG2 cells; primary human cells; peripheral blood mononuclear cells (PBMC); immune cells, T-cells; human stem cells; induced pluripotent stem cells; or somatic cells.

22. The method of claim 15, further comprising:
causing performance of transfection or transduction of one or more stable producer host cell lines or one or more packaging host cell lines for the gene therapy using one or more output models.

23. The method of claim 15, further comprising:
causing production of a viral vector in a system without transfection.

24. The method of claim 15,
wherein the plurality of data items is obtained from transient transfection, and
wherein the method further comprises:
causing development and/or production of a stable producer cell line or a packaging cell line for the gene therapy using one or more output models.

25. The method of claim 15, wherein the gene therapy includes one or more targeting moieties.

26. The method of claim 25, wherein the one or more targeting moieties comprise at least one of:
a nucleic acid sequence; a protein; a protein fragment; a peptide; a monosaccharide; a polysaccharide; a small molecule; an aptamer; a dendrimer, or a centyrin.

27. The method of claim 1, further comprising:
causing production of a nucleic acid-based therapy or vaccine for the GCT using one or more output models.

28. The method of claim 27, further comprising:
causing production of a nucleic acid for the nucleic acid-based therapy or vaccine using the one or more output models.

29. The method of claim 27, wherein the nucleic acid therapy or vaccine comprises at least one of:
DNA, plasmid DNA (pDNA), RNA, messenger RNA (mRNA), small activating RNA (saRNA), small interfering RNA (also known as short interfering RNA, silencing RNA, or siRNA), microRNA (miRNA), circular RNA, antisense oligonucleotide (ASO), doggybone DNA (dbDNA), closed-ended DNA (ceDNA), synthetic DNA, or a non-natural nucleic acid.

30. The method of claim 27, further comprising:
causing a chemical or enzymatic modification of the nucleic acid.

31. The method of claim 27, wherein the nucleic acid is combined with a non-viral carrier.

32. The method of claim 27, wherein the production comprises at least one of:
a non-viral carrier; or a physical delivery method.

33. The method of claim 27, further comprising:
causing production of one or more sequences with a plurality of nucleic acid molecules for the nucleic acid-based therapy or vaccine.

34. The method of claim 27, wherein the nucleic acid-based therapy or vaccine comprises one or more nucleic acid molecules and one or more targeting moieties.

35. The method of claim 34, wherein the one or more targeting moieties comprise at least one of:
a nucleic acid sequence; a protein; a protein fragment; a peptide; a monosaccharide; a polysaccharide; a small molecule; an aptamer; a dendrimer, or a centyrin.

36. The method of claim 27, wherein the nucleic acid-based therapy or vaccine comprises one or more nucleic acid molecules and one or more non-nucleic acid molecules.

37. The method of claim 36, wherein the one or more non-nucleic acid molecules comprise a protein, a protein fragment, or a peptide.

38. The method of claim 27, wherein the nucleic acid-based therapy or vaccine is applied to at least one of: immune cells; tumor cells; cardiac cells; ocular cells; retinal cells; lung cells; muscle cells; skin cells; liver cells; pancreatic cells; intestinal cells; brain cells; or neurological cells.

39. The method of claim 32, wherein the non-viral carrier comprises at least one of: a lipid nanoparticle; a solid lipid nanoparticle; a nanostructured lipid carrier, a liposome; a lipoplex; a polymeric nanoparticle; a lipid-polymer hybrid nanoparticle; an inorganic nanoparticle; an exosome; a virus-like particle; an extracellular vesicle; a cell-penetrating peptide; a cationic polymer; an aptamer; a dendrimer; or a centyrin.

40. The method of claim 32, wherein the physical delivery method comprises at least one of: electroporation; cell squeezing; needles; patches; iontophoresis; biolistic delivery; sonoporation; ultrasound-mediated microbubbles; hydroporation; photoporation; or magnetofection.

41. The method of claim 1, wherein the CGT comprises a cell therapy.

42. The method of claim 41, further comprising:
causing generation of one or more cells for the cell therapy based on one or more output models,
wherein the one or more cells comprise at least one of: an autologous cell; or an allogeneic cell.

43. The method of claim 41, further comprising a cell therapy created by transduction with a viral vector or transfection with a nucleic acid.

44. The method of claim 41, wherein the cell therapy is applied to at least one of: immune cells; tumor cells; cardiac cells; ocular cells; retinal cells; lung cells; pancreatic cells; intestinal cells; kidney cells; muscle cells; skin cells; liver cells; brain cells; or neurological cells.

45. The method of claim 44, wherein the cell therapy is applied to the tumor cells associated with hematological malignancies or solid tumors.

46. The method of claim 41, wherein the cell therapy comprises production of at least one of: a modified chimeric antigen receptor T-cell (CAR T-cell); a gamma delta T-cell; a natural killer (NK) cell; an engineered T-cell receptor (TCR); a tumor-infiltrating lymphocyte (TIL); a macrophage; a dendritic cell; a hematopoetic stem cell (HSC); or a mesenchymal stem/stromal cell (MSC).

47. The method of claim 41,
wherein the one or more cells comprise an autologous cell that is prepared from a source comprising at least one of: a stem cell, a pluripotent stem cell, a non-stem cell, or a cell line,
wherein the autologous cell is derived from a source comprising at least one of: peripheral blood; bone marrow; umbilical cord blood; placenta; skin; eye; muscle; or tumor.

48. The method of claim 41, wherein the one or more cells comprise an allogeneic cell that is prepared from a source comprising at least one of: peripheral blood mononuclear cells (PBMCs); umbilical cord blood; stem cells; or skin cells.

49. The method of claim 41, further comprising: causing the one or more cells to be edited.

50. The method of claim 41, further comprising: causing one or more genes in the one or more cells to be edited.

51. The method of claim 41, wherein the cell therapy comprises one or more targeting moieties.

52. The method of claim 51, wherein the one or more targeting moieties comprise at least one of:
a nucleic acid sequence; a protein; a protein fragment; a peptide; a monosaccharide; a polysaccharide; a small molecule; an aptamer; a dendrimer, or a centyrin.

53. The method of claim 41, wherein the cell therapy comprises ex vivo cell therapy.

54. The method of claim 41, wherein the cell therapy comprises at least one of: regenerative medicine; stem cell therapy; or tissue engineering.

55. The method of claim 41, wherein the cell therapy comprises in vivo cell therapy.

56. The method of claim 55, wherein the in vivo cell therapy comprises at least one of: endogenous production of a modified chimeric antigen receptor T cell (CAR T-cell); a natural killer (NK) cell; an engineered T-cell receptor (TCR); a tumor-infiltrating lymphocyte (TIL); or a macrophage.

57. The method of claim 1, wherein the CGT comprises a non-genetically modified cell therapy.

58. The method of claim 57, wherein the non-genetically modified cell therapy comprises at least one of: regenerative medicine; or tissue engineering.

59. A non-transitory computer-readable medium containing program instructions that, when executed, cause a data processing system to perform operations for developing or operating a process for a cell or gene therapy (CGT), the operations comprising:
receiving a plurality of data items;
storing the plurality of data items on a hardware storage device;
accessing the plurality of data items;
determining one or more attributes of the plurality of data items;
selecting one or more machine learning models based on the one or more attributes;
accessing one or more mechanistic models;
integrating the one or more machine learning models with the one or more mechanistic models to obtain one or more integrated models;
selecting, from the one or more machine learning models, the one or more mechanistic models, and the one or more integrated models, one or more predictive models;
applying the one or more predictive models to the plurality of data items;
adjusting one or more values of one or more parameters of the one or more predictive models to reduce uncertainty in model prediction; and
outputting the one or more predictive models with the one or more adjusted values of the one or more parameters.
